(19) United States
(12) Patent Application Publication
(10) Pub. No.: US 2025/0066731 A1
(43) Pub. Date: Feb. 27, 2025

(54) NK CELL AND PREPARATION METHOD THEREFOR

(71) Applicant: GUANGDONG PROCAPZOOM BIOSCIENCES CO., LTD., Guangzhou (CN)

(72) Inventors: Haoyu Zeng, Guangzhou (CN); Zhenbo Shen, Guangzhou (CN); Biyu Jiang, Guangzhou (CN); Yanling Zhao, Guangzhou (CN); Xiaoqing Li, Guangzhou (CN)

(21) Appl. No.: 18/944,032

(22) Filed: Nov. 12, 2024

Related U.S. Application Data
(63) Continuation of application No. PCT/CN2023/093246, filed on May 10, 2023.

(30) Foreign Application Priority Data
May 12, 2022 (CN) 202210511665.3

(51) Int. Cl. C12N 5/0783; C12N 9/22; C12N 15/11; C12N 15/86; C12N 15/90

(52) U.S. Cl. CPC C12N 5/0646; C12N 9/22; C12N 15/11; C12N 15/86; C12N 15/907; C12N 2740/15043

(57) ABSTRACT

Provided in the present disclosure is an NK cell, including a polynucleotide knocked out of CD38 and overexpressing CD16a. Compared to conventional NK cells, the NK cells of the present disclosure have been inserted with a CD16a overexpression gene and simultaneously knocked out with a CD38 gene, which improves the killing function against tumor cell lines, while simultaneously reducing the mutual killing of NK cells, improving cell survival rate and growth rate, and reducing intracellular consumption.

### CROSS-REFERENCE TO RELEATED APPLICATIONS

The present application is a Continuation Application of PCT Application No. PCT/CN2023/093246 filed on May 10, 2023, which claims priority of Chinese Patent Application No. 202210511665.3 filed on May 12, 2022 before CNIPA. All the above are hereby incorporated by reference in their entirety as part of the present disclosure.

### TECHNICAL FIELD

The present disclosure relates to the field of cellular immunotherapy and, particularly, to an NK cell with enhanced killing effect and reduced intracellular depletion and a preparation method therefor.

### BACKGROUND

Natural killer, NK, cells are important lymphocytes in the body's antiviral infection and antitumor immune response. Their source is not yet fully understood, but they are generally considered to originate from the bone marrow. Their development and maturation depend on the bone marrow microenvironment. They account for 5% to 20% of the total number of lymphocytes in peripheral blood and have a broad-spectrum antitumor effect. NK cells, as the main effector cells that kill tumours, have the characteristics of a broad spectrum of tumour killing, no need for antigen stimulation, no need for antibody participation, not restricted by MHC, and direct tumour killing.

Antibody-dependent cell mediated cytotoxicity (ADCC) refers to the direct killing of target cells by viable immune cells that identify target antigens (e.g. bacteria or tumor cells) bound by monoclonal antibodies through the binding between FC receptors and monoclonal antibodies on the cell surface. ADCC has been found to be one of the important mechanisms and means by which monoclonal antibodies can be used to treat tumours or other diseases. Among them, NK cells are the main cells mediating ADCC effects, which are stronger than those mediated by cells such as monocytes, neutrophils and macrophages.

FcγRIIIa (CD16a) belongs to the superfamily of immunoglobulins and is a low-affinity IgG receptor, a transmembrane protein expressed on the surface of immune effector cells, such as natural killer cells (NK cells), macrophages, mast cells, and neutrophils, and is a relatively important binding site for the functioning of cellular immunity in the body. CD16a binds to the Fc segment of human IgG1 and IgG3 and induces cytotoxicity of immune effector cells (mainly NK cells), i.e., ADCC, to kill target cells, or directly mediates the killing of tumour target cells by NK cells, and also promotes the secretion of cytokines, such as IFN-γ, TNF, and matrix metalloproteinases, which affect the function of cellular immunity. Monoclonal antibody-mediated molecularly targeted therapies based on the action of ADCC are becoming more widely used in malignant tumours, and CD16a as a binding site is receiving increasing attention.

It had been envisaged to overexpress CD16a on the surface of NK cells by gene transfer techniques to increase the cytotoxicity of NK cells, i.e., ADCC, or to enhance the killing effect of NK cells on tumour cells.

However, in practice, it is found that the survival rate of NK cells overexpressing CD16a is not high enough, and their killing power against tumour cells is still not strong enough.

### SUMMARY

An objective of the present disclosure is to provide an NK cell, which improves the killing function against tumor cell lines, while simultaneously reducing the mutual killing of NK cells, improving survival rate and growth rate of NK cells, and reducing intracellular consumption.

The specific technical solution of the present disclosure is as follows.

A natural killer, NK, cell includes a polynucleotide knocked out of CD38 and overexpressing CD16a.

The human CD38 antigen is a type II transmembrane glycoprotein that is expressed in various types of hematological tumor cells, but is also found on the surface of NK cells. While studying the survival rate and killing ability of NK cells expressing CD16a, it is found that after knocking out the CD38 gene, the survival rate and killing ability of NK cells overexpressing CD16a against tumour cells are both enhanced. Due to the special killing characteristics of NK cells, NK cells can also kill each other. It is theoretically speculated that knocking out the CD38 gene reduces the internal consumption between NK cells, improves the survival rate and growth rate of NK cells, and also improves the killing function against tumour cell lines.

In some implementations, the NK cell is an NK92MI cell. NK92MI cells are easier to culture than other NK cells and have high viability after transfection.

Another objective of the present disclosure is to provide a preparation method of NK cells, including the following steps:
(1) constructing a CRISPR-Cas editing system for knocking out the CD38 gene;
(2) culturing NK cells overexpressing CD16a;
(3) mixing well the CRISPR-Cas editing system of step (1) with the transfection reagent to obtain a mixture;
(4) adding the mixture of step (3) to the cultured NK cells, incubating, and continuing to culture;
(5) continuing to culture the cells obtained in step (4), sampling and testing for a negative rate; and
(6) screening for CD38-negative cells.

In some implementations, step (3) is carried out as follows: adding 250 μl of opti-MEM and 7.5 μl of lipofectamine 3000 to a first EP tube and mixing gently; adding 250 μl of opti-MEM, 15 to 100 μg of the CRISPR-Cas editing system of step (1), and 10 μl of P3000 to a second EP tube and mixing gently; and adding inclusion of the second EP tube to the first EP tube after standing for 5 min at room temperature, mixing gently, and adding to the NK cells of step (2) in equal drops after incubating for 10 to 40 min.

In some implementations, a medium employed in step (2) is a first cell culture medium, consisted by adding 0.005 to 0.6 mM of inositol, 0.00005 to 0.6 mM of folic acid, 0.0005 to 0.6mM of 2-Mercaptoethanol, 0 to 2 vol % of fetal bovine serum, 0 to 2 vol % of horse serum, and 0to 2 vol % of double antibody to a minimum essential medium, MEM.

In some implementations, a medium employed to continue to culture in step (4) is a second cell culture medium, consisted by adding 0.5 to 50 mM of inositol, 0.05 to 5 mM of folic acid, 0.05 to 5 mM of 2-Mercaptoethanol, 8 to 50 vol % of fetal bovine serum, 8 to 50 vol % of horse serum, and 0.5 to 5 vol % of double antibody to an MEM.

In some implementations, a medium employed to continue to culture in step (5) is a third cell culture medium, consisted by adding 0.05 to 5 mM of inositol, 0.005 to 0.5 mM of folic acid, 0.005 to 0.5 mM of 2-Mercaptoethanol, 5 to 20 vol % of fetal bovine serum, 5 to 20 vol % of horse serum, and 0.5 to 5 vol % of double antibody to an MEM.

Fine-tuned configuration of different culture media at different transfection and culture stages is employed in the present disclosure to culture cells in a more fine-tuned manner, rather than using the same culture medium at different stages, which can effectively improve the survival rate and transfection stability of NK cells after transfection.

In some implementations, the preparation method of NK cells overexpressing CD16a includes the following steps:
(1) establishing a lentiviral transfection system with a target plasmid having CD16a and a packaging plasmid;
(2) culturing HEK 293T cells for lentivirus and starving the HEK 293T cells (HEK 293T cells are human embryonic kidney cells that are commercially available);
(3) dividing the serum-reduced medium or serum-free medium into two equal parts, adding one part to the lentiviral transfection system with the target plasmid having CD16a, a first packaging plasmid and a second packaging plasmid, mixing well to obtain a plasmid mixture, adding the other part to the polyethyleneimine, mixing well, and then standing at room temperature for 1 to 10 min to obtain a transfection reagent;
(4) adding the transfection reagent dropwise to the plasmid mixture, mixing well, and then standing the mixture at room temperature for 5 to 40 min to obtain a mixed solution containing polyethyleneimine and the plasmid;
(5) adding the mixed solution obtained in step (4) dropwise to supernatant of a culture bottle containing HEK 293T cells, mixing well, culturing in an incubator for 6 to 9 h, aspirating and discarding supernatant, adding a cell culture medium containing 0.1 to 5 vol % of fetal bovine serum, and continuing to culture;
(6) collecting supernatant after virus transfection every 24 hours for a total of one to three times to obtain a lentiviral solution;
(7) filtering and concentrating the lentiviral solution;
(8) resuspending the cultured NK cells in the first cell culture medium, adding the concentrated lentivirus solution at a moi of 5 to 100, adding polybrene, and prewarming;
(9) centrifuging;
(10) adding the second cell culture medium containing polybrene, mixing well, and continuing to culture, in which the third cell culture medium is employed to culture and it is determined whether to semi-exchange the liquid or subculture based on medium color and cell number during culture; and
(11) taking cells for flow cytometry to determine a positive rate when cells are subcultured to more than 10 times starting number, performing flow cytometry to select positive cells when cells grow to about 5,000,000 to 10,000,000 cells, inoculating the cells into corresponding systems, continuing to amplify and culture, and establishing a library.

A variety of fine-tuned media, including a first medium, a second medium and a third medium, are employed in the preparation method of the present disclosure. In combination with the polyethyleneimine transfection reagent and the polybrene co-infectant used, as well as a specific operation method, the preparation method effectively improves the survival rate of NK cells and the overexpression efficiency of CD16a, and enables NK cells to stably overexpress CD16a even after multiple subcultures, thereby enhancing the killing effect of NK cells on tumour cells.

In some implementations, the first packaging plasmid is a psPAX2 plasmid and the second packaging plasmid is a pMD2.G plasmid, and a mass ratio of the pMD2.G plasmid, the psPAX2 plasmid, and the target plasmid having CD16a is in a range of (1-3):(1-5):(2-8). psPAX2 and pMD2.G encode HIV-1 gag-pol and vesicular stomatitis virus glycoprotein (VSV-G) respectively. The pMD2.G plasmid is an envelope plasmid that can infect a wider range of cell types, while the psPAX2 plasmid is a packaging plasmid that reduces the ability of the recombinant virus to replicate on its own, improving safety in use. Ensuring that the ratio of the three is in the range (1-3):(1-5):(2-8) can effectively improve the packaging efficiency of the entire lentivirus transfection system and lay the groundwork for achieving a high positive rate of NK cell transfection later.

In some implementations, the packaging plasmid includes a first packaging plasmid, a second packaging plasmid and a third packaging plasmid. The first packaging plasmid is a pLP1 plasmid, the second packaging plasmid is a pLP2 plasmid, the third packaging plasmid is a pLP/VSVG plasmid, the target plasmid having CD16a is an expression vector inserted with pLenti, and a mass ratio of the pLP1 plasmid, the pLP2 plasmid, the pLP/VSVG plasmid, and the target plasmid having CD16a is in a range of (1.5-7.5):(1.2-5.8):(3.2-9.3):(6.8-10). The expression vector inserted with pLenti is constructed for the insertion of the CD16a gene, which includes the y packaging signal and the truncated HIV 3′ and 5′ LTR to facilitate viral packaging. The pLP1 plasmid expresses the gag gene, which is necessary for the formation of the lentiviral structure, and the pol gene, which is necessary for viral replication and integration. The pLP1 plasmid expresses the gag gene, which is necessary for the formation of the lentiviral structure, and the pol gene, which is necessary for viral replication and integration. The pLP2 plasmid is used to express the Rev protein, which, in conjunction with the response element on pLP1, induces the expression of gag and pol and directs the nuclear transport of viral RNA. The pLP/VSVG plasmid expresses VSV-G to make the host range wider. It is only when these four plasmids work together that an infectious virus is produced. In some implementations, a ratio between polyethyleneimine and a total of the three plasmids is in a range of (1-10):1. Polyethyleneimine is employed as a transfection reagent in the present disclosure, and controlling the ratio between the polyethyleneimine and the plasmid mixture can effectively improve the virus titer and packaging efficiency of the entire lentivirus transfection system.

In some implementations, the centrifuging is carried out as follows: setting a thermostatic centrifuge to 600 to 1000 g and 30 to 36° C., and pre-centrifuging for 10 min until 30 to 36° C. is reached, transferring the pre-warmed culture plate to the centrifuge, and centrifuging at 600 to 1000 g for 30 min to 3 h at 30 to 36° C. The centrifugation method is employed to facilitate the transfecting of cells in the viral transfection system.

In some implementations, a final concentration of polybrene is in a range of 8 to 100 μg/mL.

In some implementations, the specific operation for filtering and concentrating the lentivirus solution is: filtering the supernatant after virus transfection through a 0.45-μm filter; adding 5 to 10 ml of PEG-6000 NaCl mother liquor to every 10 to 80 ml of filtered initial lentivirus solution, standing at 4° C., mixing and shaking well every 20 to 30 minutes for a total of 3 to 5 times; standing overnight at 4° C.; centrifuging at 4° C. for 10 to 40 minutes at 4,000 to 6,000 g; aspirating and discarding the supernatant, standing for one to two minutes, removing any remaining liquid, adding an appropriate amount of lentivirus dissolving solution to dissolve the lentivirus precipitate, mixing well, and dividing the mixture into aliquots. The virus titer of lentivirus can be increased by employing this filtration and concentration method.

In another aspect, provided in the present disclosure is further an NK cell knocked out of CD38 and overexpressing Cd16a prepared by the aforementioned preparation method.

The NK cells knocked out of the CD38 gene and overexpressing CD16a in the present disclosure exhibit enhanced survival rate and killing performance against tumour cells.

At the same time, a variety of fine-tuned media are employed in the preparation method of the present disclosure. In combination with the polyethyleneimine transfection reagent and the polybrene transfection enhancer used, as well as a specific operation method, the preparation method effectively improves the survival rate of NK cells, the negative rate of CD38, and the overexpression efficiency of CD16a, and enables NK cells to stably overexpress CD16a even after multiple subcultures, thereby reducing NK cell self-killing and enhancing the killing effect of NK cells on tumour cells.

### BRIEF DESCRIPTION OF THE DRAWINGS

FIG. 1 is a flow cytometry diagram of the NK92MI cell clone 1 constructed by example II, overexpressing CD16a and knocked out CD38.

FIG. 2 is a flow cytometry diagram of the NK92MI cell clone 1 after being cultured for 2 months constructed by example II, overexpressing CD16a and knocked out CD38.

FIG. 3 is a flow cytometry diagram of the NK92MI cell clone 2 constructed by example III, overexpressing CD16a and knocked out CD38.

FIG. 4 is a flow cytometry diagram of the NK92MI cell clone 2 after being cultured for 2 months constructed by example III, overexpressing CD16a and knocked out CD38.

FIG. 5 is a flow cytometry diagram of the NK92MI cell clone 3 constructed by contrast example I, overexpressing CD16a and knocked out CD38.

FIG. 6 is a flow cytometry diagram of the NK92MI cell clone 3 after being cultured for 2 months constructed by contrast example I, overexpressing CD16a and knocked out CD38.

FIG. 7 is a flow cytometry diagram of the NK92MI cell clone 4 constructed by contrast example II, overexpressing CD16a and knocked out CD38.

FIG. 8 is a flow cytometry diagram of the NK92MI cell clone 4 after being cultured for 2 months constructed by contrast example II, overexpressing CD16a and knocked out CD38.

FIG. 9 is a flow cytometry diagram of the NK92MI cell clone 5 constructed by contrast example III, overexpressing CD16a and knocked out CD38.

FIG. 10 is a flow cytometry diagram of the NK92MI cell clone 5 after being cultured for 2 months constructed by contrast example III, overexpressing CD16a and knocked out CD38.

FIG. 11 is a flow cytometry diagram of the NK92MI cell clone 6 constructed by contrast example IV, overexpressing CD16a but without knocking out CD38;

FIG. 12 is a flow cytometry diagram of the NK92MI cell clone 6 constructed by contrast example IV after being cultured for 2 months, overexpressing CD16a but without knocking out CD38.

FIG. 13 is a statistical chart of the lethality of multiple clones of NK92MI cells knocked out of CD38 and overexpressing CD16a against different tumour cells.

### DETAILED DESCRIPTION

For a better understanding of the solutions of the present disclosure by those skilled in the art, the technical solutions in the examples of the present disclosure are clearly and completely described and discussed below. Obviously, the examples described herein are only some of the examples of the present disclosure but not all of them.

It should be understood that the various aspects described below are not limited to specific compositions, methods for preparing the compositions, or uses thereof, as these aspects may of course change before the present disclosure is disclosed and described.

It should also be understood that the terms used in the present disclosure are intended solely to describe specific aspects and are not intended to be limiting.

Unless otherwise defined, all terms including technical and scientific terms used herein have the same meaning as commonly understood by those skilled in the art to which the present disclosure belongs.

The terms used in the present disclosure are intended solely to describe specific examples and are not intended to limit the present disclosure. As used in the present disclosure, unless the context clearly indicates otherwise, the singular forms “a”, “an” and “the” also include the plural forms. All numerical values, for example, pH, temperature, time, concentration, dosage and molecular weight, including ranges, are approximate values that may change by an increase or a decrease of 10%, 1% or 0.1%, if applicable. It should be understood that although it is not always explicitly stated, the term “approximately” can be added in front of all values. It should also be understood that, although not always expressly indicated, the reagents described in the present disclosure are exemplary only, and that equivalent reagents to the described reagents are known in the art.

“Optional” or “optionally” means that the event or circumstance described subsequently may or may not occur, and the description covers both cases where the event or circumstance occurs and where it does not.

The terms “include” and other derivatives thereof are intended to mean that the composition or method includes the elements listed but does not exclude others. The term “consisting essentially of”' as used in definitions of composition and method is to be understood as excluding any element that is essential to the composition. For example, a composition consisting essentially of the elements defined in the present disclosure does not exclude other elements that do not substantially affect the essential new characteristics of the present disclosure. “Consisting of” should be understood to exclude other components and substantial method steps that are listed in excess of trace amounts. These transitional term definition examples are within the scope of the present disclosure.

As used herein, the term “overexpression” refers to a level of CD16a in the cell (e.g., expression level) that significantly exceeds a level in the initial cell (a cell that has not been transduced with the foreign gene); e.g., 20%, preferably 50%, more preferably more than 100%, e.g., 200%, 300%, 500% or more, as compared to the level in the initial cell. An example of “overexpression” is the transfer and expression of a coding gene for an exogenous transcription factor into a cell.

In the present disclosure, the NK cells described can be isolated from the body, including autologous and allogeneic NK cells. The NK cells described can be cultured in vitro and can be primary cultures or passaged cultures. Nowadays, there are also commercially available NK cells that can be easily obtained by those skilled in the art, for example, NK92MI, a natural killer cell for patients with human malignant non-Hodgkin's lymphoma, which can be obtained from ATCC (ATCC CRL-2408). Furthermore, other established NK cell lines in the art include such as NK92, NKL, YT, HANK-1, NK-YS, and SNK-6. It should be understood that all of them can be used in the present disclosure.

The term “lentiviral vector” refers to a viral vector or plasmid that contains structural and functional genetic elements predominantly of lentiviral origin or parts thereof.

The terms “lentiviral vector” or “lentiviral expression vector” may be used to refer to a lentiviral transfer plasmid and/or an infected lentiviral particle. It should be understood that nucleic acid sequence elements (such as cloning sites, promoters, regulatory elements, and heterologous nucleic acids) are present in the form of RNA in the lentiviral particles of the present disclosure, and in the form of DNA in the DNA plasmids of the present disclosure.

### EXAMPLE I: PREPARATION OF NK916 CELLS (NK92MI CELLS OVEREXPRESSING CD16)

(1) A three-plasmid system was prepared, consisting of the target plasmid overexpressing CD16, the psPAX2 plasmid, and the pMD2.G plasmid.

(2) A basic medium was prepared: Dulbecco's modified Eagle's medium (DMEM) complete medium containing 5 to 20 vol % fetal bovine serum and 1 vol % penicillin-streptomycin.

(3) Approximately one to ten million 293T cells were inoculated into a T75 culture bottle, basic medium was added, and the cells were incubated overnight. When the cell density reached 50 to 90%, the supernatant in the culture bottle was aspirated and discarded, and 10 mL of DMEM medium containing only 0.1 to 2 vol % fetal bovine serum was added to starve the cells for 1 to 3 h to obtain tool cells.

(4) Opti-MEM medium was aspirated in an amount of 1 to 10 mL per virus and divided equally into two equal portions. One portion was added with the lentiviral packaging plasmids psPAX2, pMD2.G and the target expression plasmid, mixed well to obtain a plasmid mixture; the other portion was added with polyethyleneimine, mixed well, and then stood at room temperature for 1 to 10 minutes to obtain a transfection reagent; a mass ratio of pMD2.G, psPAX2 and the target expression plasmid was 1:1:8, and a mass ratio of polyethyleneimine to a total mass of the three plasmids was 10:1.

(5) The transfection reagent was added dropwise to the plasmid mixture, after being mixed well, and then the mixture was stood at room temperature for 5 to 40 min to obtain a mixed solution containing polyethyleneimine and the plasmid.

(6) The mixed solution obtained in step (5) was added dropwise to the supernatant of the culture bottle containing the tool cells. The pipette tip should be suspended in the air during the addition, and the movement should be gentle. Be careful not to blow up the cells. After adding, the bottle should be gently shaken according to the cross method to mix the viral packaging components and the medium thoroughly, and then placed in an incubator for 6 to 9 hours of culture. The supernatant should be aspirated and discarded, and 5 to 20 mL of fresh pre-warmed DMEM medium containing 0.1 to 5 vol % fetal bovine serum should be added to each bottle for a further 72 hours of culture.

(7) The supernatant was collected every 24 hours after virus transfection and filtered through a 0.45-μm filter, starting from the addition of fresh DMEM medium containing 0.1 to 5 vol % fetal bovine serum. The medium was replaced with fresh DMEM medium containing 0.1 to 5 vol % fetal bovine serum. A total of 3 times was collected and stored at 4° C. temporarily.

(8) 7.5 ml of PEG-6000 NaCl mother liquor was added to every 10 to 80 ml of filtered initial virus liquid. It was stood at 4° C., mixed and shaken well every 20 to 30 minutes for a total of 3 to 5 times. Then, it was stood overnight at 4° C. Finally, it was centrifuged at 4° C. for 10 to 40 minutes at 4,000 to 6,000 g. The PEG-6000 NaCl mother liquor is prepared as follows: 20 to 40 g of NaCl and 100 to 300 g of PEG 6000 was dissolved into 500 ml of ultrapure water; after dissolving, the liquid was placed in a 121° C. autoclave for 30 minutes; and the sterilized mother liquor was filtered through a 0.45-um filter and stored at 4° C.

(9) The supernatant was aspirated and discarded, the tube was allowed to stand for 1 to 2 minutes, any remaining liquid was removed, and the lentivirus precipitate was dissolved by adding an appropriate amount of lentivirus dissolving solution (DMEM basic medium). The mixed and dissolved virus suspension was aliquoted to obtain the NK916 lentivirus, stored at −80° C., and used as needed.

(10) An appropriate amount of NK92MI cell suspension with good growth status was taken, centrifuged at 300 g for 2 to 5 min, the supernatant was removed, the NK92MI cells were collected, the cells were resuspended in the first culture medium, and fifty thousand to five million cells (not more than 1 mL) were added to each well of a 12-well culture plate. The NK916 lentivirus was added to the 12-well culture plate at an MOI of 5 to 100. Polybrene (final concentration 100 g/mL) was added, followed by the addition of first culture medium up to a total volume of 3 mL. The culture plate was placed in a 37° C. incubator for 10 min to preheat.

(11) Meanwhile, the thermostat of the centrifuge was set to 600 to 1000 g, 30 to 36° C., and it was centrifuged for 10 min to preheat to 30 to 36° C. The preheated 12-well culture plate was transferred to the centrifuge and centrifuged at 600 to 1000 g for 30 min to 3 h at 30 to 36° C.

(12) After centrifugation, the 12-well culture plate was removed and 1 mL of the second culture medium containing polyamine (final concentration of polyamine was 100 μg/mL) was added to each well. The pipette tip was gently tapped against the center of each well three to five times to mix the solution. The culture plate was transferred back to the incubator for culture. During the culture period, the medium was replaced or cells were subcultured according to the medium color and cell number, and the culture medium was the third culture medium.

(13) 200,000 cells were taken for flow cytometry to determine a positive rate when cells are subcultured to more than 10 times the starting number. Cells were prepared to perform flow cytometry to select positive cells when cells grow to about 5,000,000 to 10,000,000 cells, inoculating the cells into corresponding systems, continuing to amplify and culture, and establishing a library.

The first cell culture medium was consisted by adding 0.5 mM of inositol, 0.5 mM of folic acid, 0.05 mM of 2-Mercaptoethanol, 2 vol % of fetal bovine serum, 2 vol % of horse serum, and 2 vol % of double antibody to an MEM.

The second cell culture medium was consisted by adding 1 mM of inositol, 0.5 mM of folic acid, 0.1 mM of 2-Mercaptoethanol, 20 vol % of fetal bovine serum, 30 vol % of horse serum, and 1 vol % of double antibody to an MEM.

The third cell culture medium was consisted by adding 2 mM of inositol, 0.1 mM of folic acid, 0.005 mM of 2-Mercaptoethanol, 20 vol % of fetal bovine serum, 5 vol % of horse serum, and 0.5 vol % of double antibody to an MEM.

### EXAMPLE II: PREPARATION OF NK986 CELLS (NK92MI CELLS KNOCKED OUT CD38 AND OVEREXPRESSING CD16)

On the basis of example 1, the following steps were performed:

(14) A CD38-deficient gRNA expression vector was constructed using CRISPR: a guide RNA (5′-TGAACTCGCAGTTGGCCATA-3′) target the CD38 gene was designed to form a complex with the Cas9 nuclease, and this complex will knock out CD38 when transduced into cells.

(15) The NK916 cells prepared in Example 1 were inoculated into two wells of a six-well culture plate in terms of a hundred thousand to a million cells/well, and 2 mL of the first cell culture medium was added thereto and cultured for 2 to 6 h.

(16) First EP tube: 250 μl of opti-MEM +7.5 μl of lipofectamine 3000, mixed gently; second EP tube: 250 μl of opti-MEM +15-100 μg of target plasmid +10 μl of lipofectamine 3000, mixed gently. The inclusion of the second EP tube was added to the first EP tube after being stood for 5 min at room temperature, mixed gently, and adding to the NK cells of step (2) in equal drops after incubating for 10 to 40 min.

(17) 1 mL of the second cell culture medium was added after 3 to 12 h for further culture.

(18) From the next day, the cells were cultured in the third cell culture medium until the starting number was about five times the initial number. 200,000 cells were sampled for flow cytometry to test the negative rate. When the number of cells has reached about five million to ten million, the cells were incubated with the antibody and then sorted using flow cytometry to remove the CD38-negative cells.

The first, second and third cell culture media employed in example 2 were the same as those in example 1.

Compared to NK92MI cells, NK986 cells were modified by inserting the CD16 overexpression gene and knocking out the CD38 gene.

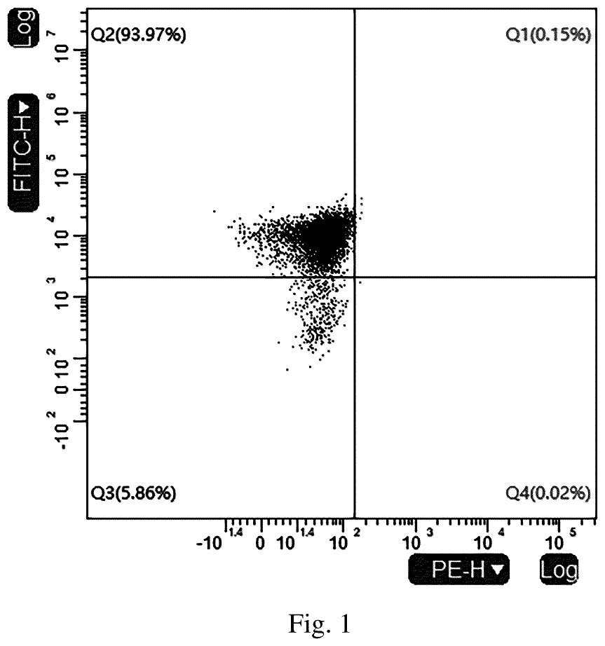

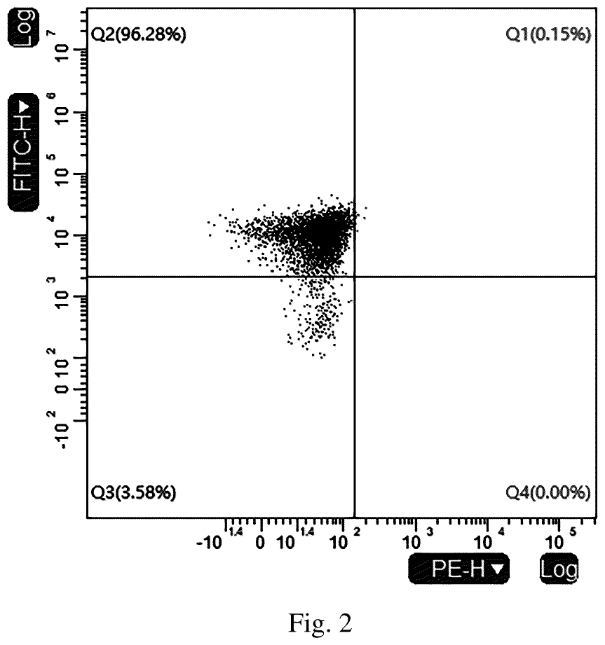

The flow cytometry diagram of the NK92MI cell clone 1 constructed by example II, overexpressing CD16a and knocked out CD38, is shown in FIG. 1. The flow cytometry diagram of the NK92MI cell clone 1 constructed by example II, overexpressing CD16a and knocked out CD38, after being cultured for 2 months is shown in FIG. 2.

In the flow cytometry diagram, FITC-H is an indicator of CD16a and PE-H is an indicator of CD38. It can be seen that the areas representing CD38 in Q1 and Q4 are both empty, and the coordinate axis represents the amount of specific antigen expressed by each cell. The more points in Q2 represent a greater degree of overexpression of CD16a.

### EXAMPLE III

The operation of example III was basically the same as that of a combination of example I and example II. The main difference was that the first, second and third media in example I and example II were replaced as follows; the mass ratio of pMD2.G, psPAX2 and the target expression plasmid was 3:5:2, the total mass ratio of polyethyleneimine to the three plasmids was 1:1, and the final concentration of polybrene was 8 μg/mL.

The first cell culture medium was consisted by adding 0.1 mM of inositol, 0.2 mM of folic acid, 0.1 mM of 2-Mercaptoethanol, 2 vol % of fetal bovine serum, 2 vol % of horse serum, and 2 vol % of double antibody to an MEM.

The second cell culture medium was consisted by adding 1 mM of inositol, 0.05 mM of folic acid, 0.1 mM of 2-Mercaptoethanol, 25 vol % of fetal bovine serum, 25 vol % of horse serum, and 1 vol % of double antibody to an MEM.

The third cell culture medium was consisted by adding 0.05 mM of inositol, 0.05 mM of folic acid, 0.1 mM of 2-Mercaptoethanol, 10 vol % of fetal bovine serum, 10 vol % of horse serum, and 5 vol % of double antibody to an MEM.

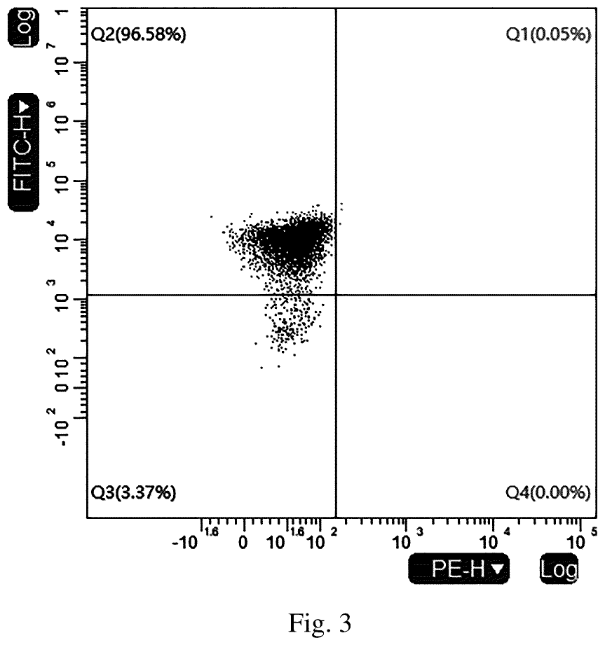

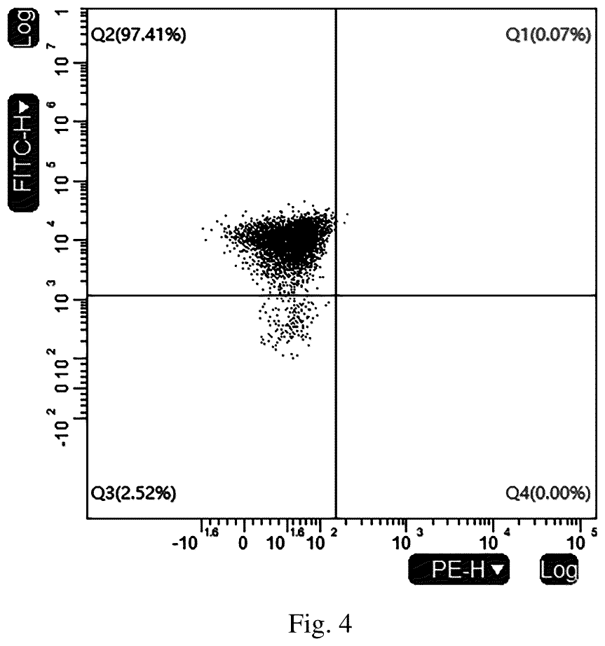

The flow cytometry diagram of the NK92MI cell clone 2 constructed by example III, overexpressing CD16a and knocked out CD38, is shown in FIG. 3. The flow cytometry diagram of the NK92MI cell clone 2 constructed by example III, overexpressing CD16a and knocked out CD38, after being cultured for 2 months is shown in FIG. 4.

### CONTRAST EXAMPLE I

The operation of contrast example I was basically the same as that of example I and example II. The main difference was that the first, second and third media in example I and example II were replaced as follows, and the final concentration of polyamine was 15 μg/mL.

The first cell culture medium was consisted by adding 0.05 mM of folic acid, 0.05 mM of 2-Mercaptoethanol, 1 vol % of fetal bovine serum, 2 vol % of horse serum, and 1 vol % of double antibody to an MEM.

The second cell culture medium was consisted by adding 2 mM of folic acid, 0.5 mM of 2-Mercaptoethanol, 15 vol % of fetal bovine serum, 30 vol % of horse serum, and 15 vol % of double antibody to an MEM.

The third cell culture medium was consisted by adding 0.5 mM of folic acid, 0.5 mM of 2-Mercaptoethanol, 10 vol % of fetal bovine serum, 5 vol % of horse serum, and 15 vol % of double antibody to an MEM.

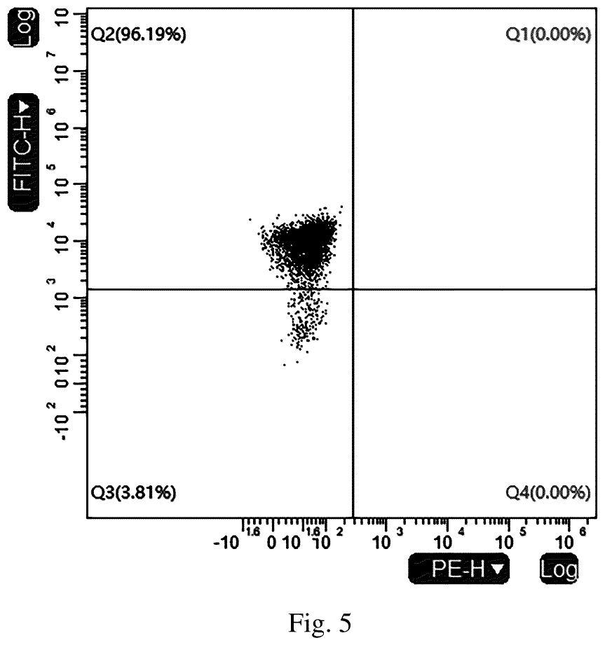

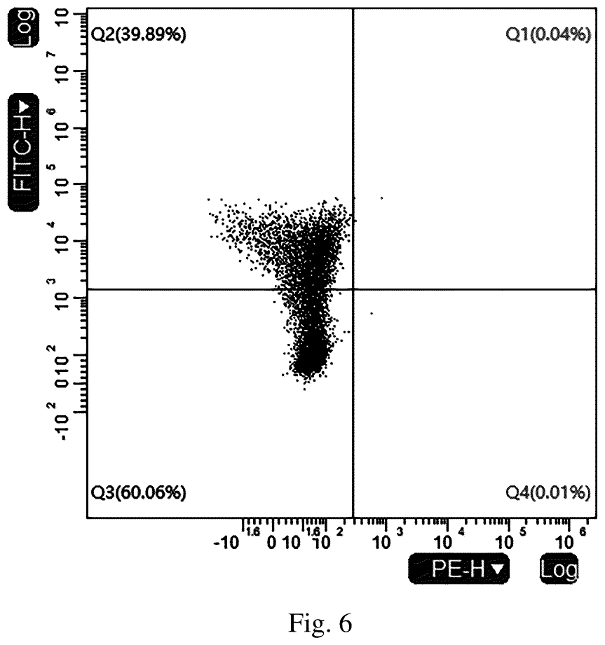

The flow cytometry diagram of the NK92MI cell clone 3 constructed by contrast example I, overexpressing CD16a and knocked out CD38, is shown in FIG. 5. The flow cytometry diagram of the NK92MI cell clone 3 constructed by contrast example I, overexpressing CD16a and knocked out CD38, after being cultured for 2 months is shown in FIG. 6.

### CONTRAST EXAMPLE II

The operation of contrast example II was basically the same as that of example I and example II. The main difference was that the first, second and third media in example I and example II were replaced as follows; the mass ratio of pMD2.G, psPAX2 and the target expression plasmid was 3:5:2, the total mass ratio of polyethyleneimine to the three plasmids was 10:1, and the final concentration of polybrene was 20 μg/mL.

The first cell culture medium was consisted by adding 0.6 mM of inositol, 0.6 mM of 2-Mercaptoethanol, 2 vol % of fetal bovine serum, 1 vol % of horse serum, and 1 vol % of double antibody to an MEM.

The second cell culture medium was consisted by adding 0.5 mM of inositol, 1 mM of 2-Mercaptoethanol, 40 vol % of fetal bovine serum, 10 vol % of horse serum, and 1 vol % of double antibody to an MEM.

The third cell culture medium was consisted by adding 0.1 mM of inositol, 0.5 mM of 2-Mercaptoethanol, 5 vol % of fetal bovine serum, 5 vol % of horse serum, and 1 vol % of double antibody to an MEM.

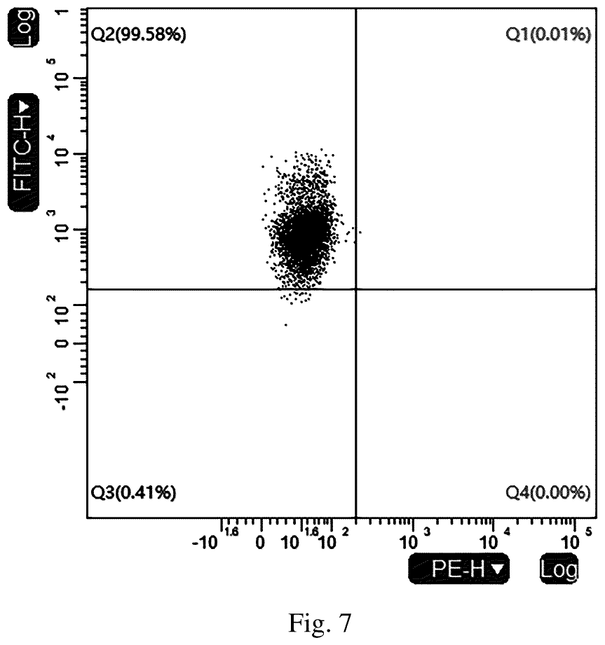

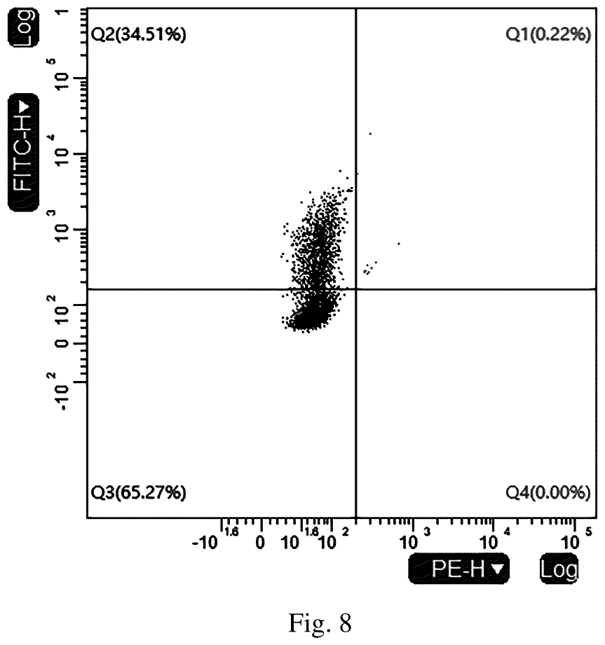

The flow cytometry diagram of the NK92MI cell clone 4 constructed by contrast example II, overexpressing CD16a and knocked out CD38, is shown in FIG. 7. The flow cytometry diagram of the NK92MI cell clone 4 constructed by contrast example II, overexpressing CD16a and knocked out CD38, after being cultured for 2 months is shown in FIG. 8.

### CONTRAST EXAMPLE III

The operation of contrast example III was basically the same as that of example I and example II. The main difference was that the first, second and third media in example I and example II were replaced as follows; the mass ratio of pMD2.G, psPAX2 and the target expression plasmid was 3:5:8, the total mass ratio of polyethyleneimine to the three plasmids was 5:1, and the final concentration of polybrene was 50 μg/mL.

The first cell culture medium was consisted by adding 0.01 mM of inositol, 0.05 mM of folic acid, 2 vol % of fetal bovine serum, 1 vol % of horse serum, and 1 vol % of double antibody to an MEM.

The second cell culture medium was consisted by adding 0.5 mM of inositol, 0.05 mM of folic acid, 30 vol % of fetal bovine serum, 30 vol % of horse serum, and 2 vol % of double antibody to an MEM.

The third cell culture medium was consisted by adding 0.1 mM of inositol, 0.05 mM of folic acid, 4 vol % of fetal bovine serum, 6 vol % of horse serum, and 2 vol % of double antibody to an MEM.

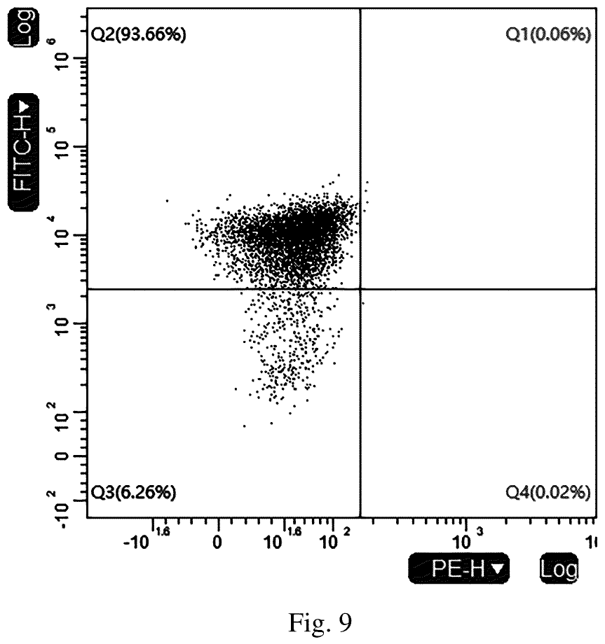

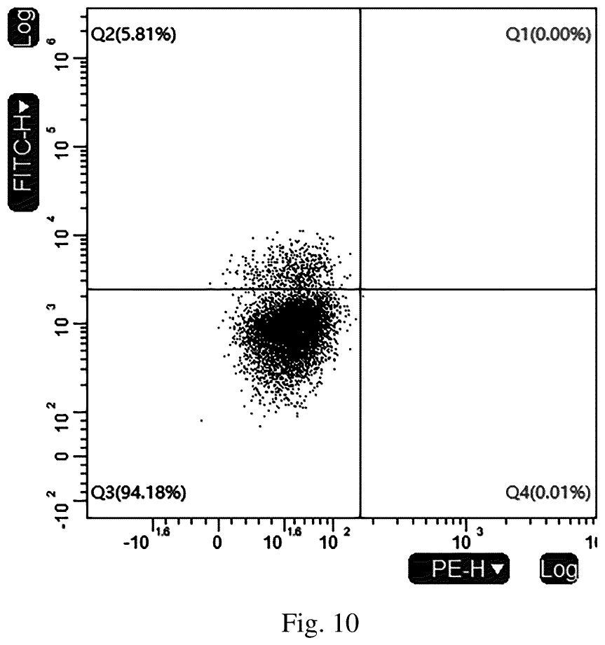

The flow cytometry diagram of the NK92MI cell clone 5 constructed by contrast example III, overexpressing CD16a and knocked out CD38, is shown in FIG. 9. The flow cytometry diagram of the NK92MI cell clone 5 constructed by contrast example III, overexpressing CD16a and knocked out CD38, after being cultured for 2 months is shown in FIG. 10.

### CONTRAST EXAMPLE IV

The contrast example IV was performed the same way as the example I, but without the steps of example II, i.e. the contrast example IV is only the NK cells overexpressing CD16a, without knocking out the CD38 gene.

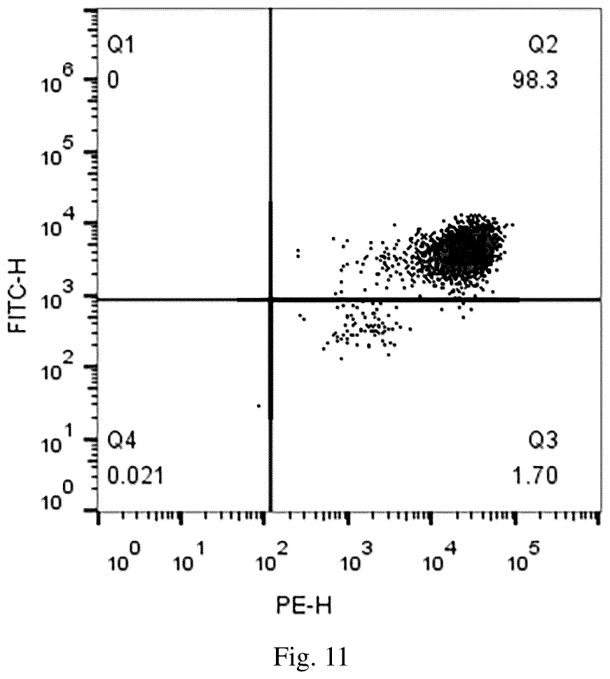

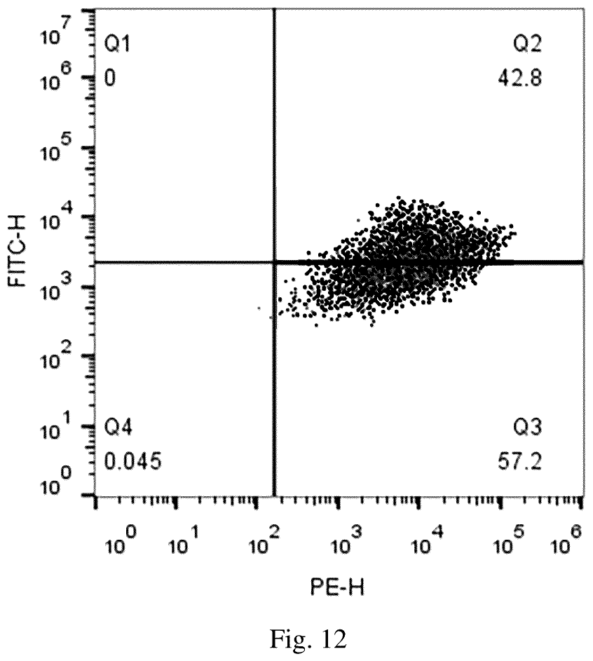

The flow cytometry diagram of the NK92MI cell clone 6 constructed by contrast example IV, overexpressing CD16a but without knocking out CD38, is shown in FIG. 11. The flow cytometry diagram of the NK92MI cell clone 6 constructed by contrast example IV, overexpressing CD16a but without knocking out CD38, after being cultured for 2 months is shown in FIG. 12.

### Test: Negative/Positive Rate Detection

The test subjects in the present test example were NK cells knocked out of CD38 and overexpressing CD16a prepared in examples II to III and contrast examples I to IV, which were de-virused and sampled for testing using a flow cytometer.

(a) CD38: Incubation of anti-CD38+PE streptomycin

(b) CD16: Direct FITC detection

### Detailed Steps

1. Two flow tubes were taken, and according to the counting results, about 100,000 to 500,000 cells were added to each tube for the control group and the experimental group.

2. The flow tube was centrifuged at 25° C. for 3 to 10 minutes at 200 g in a centrifuge.

3. The flow tube was taken out and the supernatant was discarded.

4. 100 to 300 μL of saline and 5 to 10 μL of anti-CD38 antibody were added to each tube and incubated at 2 to 8° C. for 30 to 60 min.

5. The flow tube was taken out, 1 ml of saline was added to each sample tube, the mixture was centrifuged at 200 g for 5 minutes, washed once, and the supernatant was discarded.

6. 100 to 300 μL of saline was added to each tube to resuspend the cells, 5 to 10 μL of PE streptomycin secondary antibody was added, and the mixture was incubated at 2 to 8° C. for 30 to 60 min.

7. The flow tube is taken out, 1 ml of saline is added to each sample tube, mixed well, placed in a centrifuge at 200 g for 3 to 10 minutes at 20° C., the flow tube is taken out, the supernatant is discarded, and the washing is repeated once.

8. The supernatant was discarded, 500 μl of saline was added to each tube, and the mixture was mixed by vortexing.

9. FITC and PE were detected on the machine, and a cross gate was drawn according to the control group. The cross gate consisted of a CD16 gate (the gate was a horizontal line at the top of the control group cell population) and a CD38 gate (the gate was a vertical line on the left side of the control group). The proportion of CD16+and CD38-target cell populations in the experimental group was analyzed (the proportion of cells in the upper left of the cross gate to all cells in the cross gate).

The detection results of the flow cytometer are shown in FIGS. 1, 3, 5, 7, 9 and 11. As can be seen, the positive rate of NK cells initially transfected was quite high, all of which reached above 90%, and the negative rate of CD38 is stable.

The negative rate was detected again using flow cytometry after these transfected cells had been subcultured for two months. The detection results of the flow cytometer are shown in FIGS. 2, 4, 6, 8, 10 and 12. As can be seen, other than the cell clones in Examples II and III, which stably and highly express CD16a, the cell clones in the other examples have all undergone different degrees of off-targeting. Especially in contrast examples III and IV, after two months of culture, the positive rate fell below 5%. However, NK cells in examples I and II were still able to stably and efficiently overexpress CD16a after two months of culture.

### Test: Lethality Assessment

The NK cells (called NK986 cells) that had been knocked out of CD38 and overexpressed CD16a in examples II to III and contrast examples I to III were cultured for two months and then tested for their killing ability in the following way:

1. Plate washing: The liquid was sucked out of the plate, and each hole was washed twice with autoclaved water. The plate was then immersed in a NaOH solution for one to four hours, washed twice with autoclaved water, and finally rinsed once with DPBS.

2. Plating: The cultured target tumor cell lines (BxPC-3, OVCAR-3, AGS, and HO8910 tumor cells) were digested, centrifuged, resuspended, stained with Trypan blue, and counted. The cells were then inoculated into a current plate at a density of 10,000 to 100,000 live cells per well, supplemented with an appropriate amount of medium, and placed in an incubator for monitoring. If the cell growth curve rises smoothly and there is not much difference between the wells, then the cell growth is considered normal.

3. NK cells knocked out of CD38 and overexpressing CD16a prepared by examples II to III and contrast examples I to III were added: the beginning of the growth curve plateau of the tumor cell line was taken as the time point of adding NK cells. The NK cells to be tested were removed, centrifuged, resuspended, stained with Trypan blue and counted. They were added to the current plate at a ratio of tumor cell inoculum: NK cell=3:1. The machine (real-time cell analyzer) was placed in an incubator to continuously monitor the killing. Untransfected NK92MI cells were used as a negative control, and NK92MI cells that were not knocked out of CD38 and only overexpressed CD16a (NK916 cells) were used as a control.

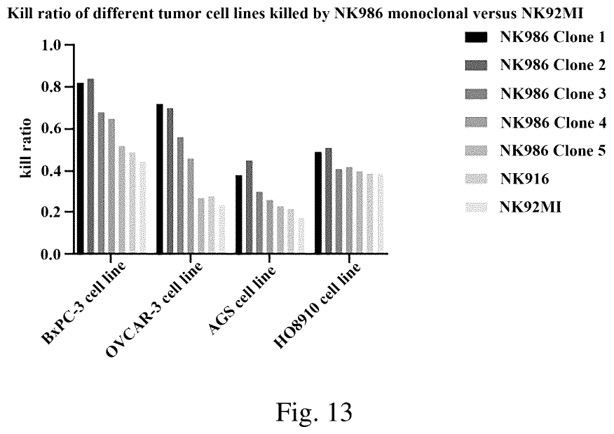

As can be seen in FIG. 13, NK cells knockout CD38 and overexpressing CD16a in both example II and III have significantly enhanced killing performance for these four tumor cells relative to the negative control and the 916 cell line without being knocked out CD38. The killing ability of NK cells knocked out of CD38 and overexpressing CD16a against the four tumor cells in contrast examples I to III was also enhanced to some extent, but the degree of enhancement was significantly lower than that in examples II and III. The killing ability of untransfected NK92MI cells and NK92MI cells overexpressing only CD16a without being knocked out of CD38 (NK916 cells) was even lower.

The above examples are only used to illustrate the technical solution of the present disclosure rather than to limit the protection scope of the present disclosure. Although the present disclosure has been described in detail with reference to the above examples, a person of ordinary skill in the art should be understood that modifications or equivalent substitutions may be carried out to the technical solution of the present disclosure without departing from the substance and scope of the technical solution of the present disclosure.

1. A preparation method of natural killer, NK, cells, the NK cells comprising a polynucleotide knocked out of CD38 and overexpressing CD16a, the NK cells being NK92MI cells, wherein the preparation method comprises following steps:
(1) constructing a CRISPR-Cas editing system for knocking out CD38 gene;
(2) culturing NK cells overexpressing CD16a;
(3) mixing well the CRISPR-Cas editing system of step (1) with a transfection reagent to obtain a mixture;
(4) adding the mixture of step (3) to the cultured NK cells, incubating, and continuing to culture;
(5) continuing to culture the cells obtained in step (4), sampling and testing for a negative rate; and
(6) screening for CD38-negative cells,
wherein a medium employed in step (2) is a first cell culture medium, consisted by adding 0.5 mM of inositol, 0.5 mM of folic acid, 0.05 mM of 2-Mercaptoethanol, 2 vol % of fetal bovine serum, 2 vol % of horse serum, and 2 vol % of double antibody to a minimum essential medium, MEM,
wherein a medium employed to continue to culture in step (4) is a second cell culture medium, consisted by adding 1 mM of inositol, 0.5 mM of folic acid, 0.1 mM of 2-Mercaptoethanol, 20 vol % of fetal bovine serum, 30 vol % of horse serum, and 1 vol % of double antibody to an MEM wherein a medium employed to continue to culture in step (5) is a third cell culture medium, consisted by adding 2 mM of inositol, 0.1 mM of folic acid, 0.005 mM of 2-Mercaptoethanol, 20 vol % of fetal bovine serum, 5 vol % of horse serum, and 0.5 vol % of double antibody to an MEM.

2. The preparation method of NK cells according to claim 1, wherein step (3) is carried out as follows:
adding 250 μL of opti-MEM and 7.5 μL of lipofectamine 3000 to a first EP tube and mixing gently;
adding 250 μL of opti-MEM, 15 to 100 μg of the CRISPR-Cas editing system of step (1), and 10 μL of P3000 to a second EP tube and mixing gently; and
adding mixture in the second EP tube to the first EP tube after standing for 5 min at room temperature, mixing gently, and adding mixture to the NK cells of step (2) in equal drops after incubating for 10 to 40 min.

3. The preparation method of NK cells according to claim 1, wherein NK cells overexpressing CD16a in step (2) are prepared by following steps:
(1) establishing a lentiviral transfection system with a target plasmid having CD16a and a packaging plasmid;
(2) culturing HEK 293T cells for lentivirus and starving the HEK 293T cells;
(3) dividing a serum-reduced medium into two equal parts, adding one part to the lentiviral transfection system with the target plasmid having CD16a, a first packaging plasmid and a second packaging plasmid, mixing well to obtain a plasmid mixture, adding polyethyleneimine to the other part, mixing well, and then standing at room temperature for 1 to 10 min to obtain a transfection reagent;
(4) adding the transfection reagent dropwise to the plasmid mixture, mixing well, and then standing the mixture at room temperature for 5 to 40 min to obtain a mixed solution containing polyethyleneimine and the plasmid;
(5) adding the mixed solution obtained in step (4) dropwise to supernatant of a culture bottle containing HEK 293T cells, mixing well, culturing in an incubator for 6 to 9 h, aspirating and discarding supernatant, adding a cell culture medium containing 0.1 to 5 vol % of fetal bovine serum, and continuing to culture;
(6) collecting supernatant after virus transfection every 24 hours for a total of one to three times to obtain a lentiviral solution;
(7) filtering and concentrating the lentiviral solution;
(8) resuspending the cultured NK cells in the first cell culture medium, adding the concentrated lentivirus solution at a moi of 5 to 100, adding polybrene, and prewarming;
(9) centrifuging;
(10) adding the second cell culture medium containing polybrene, mixing well, and continuing to culture, in which the third cell culture medium is employed to culture and it is determined whether to semi-exchange liquid or subculture based on medium color and cell number during culture; and
(11) taking cells for flow cytometry to determine a positive rate when cells are subcultured to more than 10 times starting number, performing flow cytometry to select positive cells when cells grow to 5,000,000 to 10,000,000 cells, inoculating the cells into corresponding systems, continuing to amplify and culture, and establishing a library.

4. The preparation method of NK cells according to claim 3, wherein the packaging plasmid comprises a first packaging plasmid and a second packaging plasmid, the first packaging plasmid is a psPAX2 plasmid and the second packaging plasmid is a pMD2.G plasmid, and a mass ratio of the pMD2.G plasmid, the psPAX2 plasmid, and the target plasmid having CD16a is in a range of (1-3):(1-5):(2-8); or,
wherein the packaging plasmid comprises a first packaging plasmid, a second packaging plasmid and a third packaging plasmid, the first packaging plasmid is a pLP1 plasmid, the second packaging plasmid is a pLP2 plasmid, the third packaging plasmid is a pLP/VSVG plasmid, the target plasmid having CD16a is an expression vector inserted with pLenti, and a mass ratio of the pLP1 plasmid, the pLP2 plasmid, the pLP/VSVG plasmid, and the target plasmid having CD16a is in a range of (1.5-7.5):(1.2-5.8):(3.2-9.3):(6.8-10).

5. The preparation method of NK cells according to claim 4, wherein a ratio between polyethyleneimine and a total of the plasmids containing packaging plasmids and target plasmid is in a range of (1-10):1.
